(19) United States
(12) Patent Application Publication
(10) Pub. No.: US 2005/0159724 A1
(43) Pub. Date: Jul. 21, 2005

(54) Needleless access vial

(76) Inventor: Jon R. Enerson, Port Richey, FL (US)

Correspondence Address: HOLLAND & KNIGHT LLP, ATTN: STEFAN V. STEIN/ IP DEPT., POST OFFICE BOX 1288, TAMPA, FL, 33601-1288, US

(21) Appl. No.: 11/019,002

(22) Filed: Dec. 20, 2004

Related U.S. Application Data
(60) Provisional application No. 60/531,027, filed on Dec. 18, 2003.

(51) Int. Cl. A61M 5/32; A61B 19/00

(52) U.S. Cl. 604/411

(57) ABSTRACT

A needleless access vial, including a vial, a valve for sealing the vial, the valve comprising a valve element positioned in a valve body, a retainer positioned about the valve body, valve element and a portion of the vial to sealingly sandwich the valve element between a portion of the vial and the valve body and the valve element comprising a central passageway including a slit formed therein for receiving a neck of an access device such as a syringe.

### CROSS-REFERENCE TO RELATED APPLICATIONS

This application claims the benefit of provisional application 60/531,027 filed Dec. 18, 2003, the disclosure of which is hereby incorporated by reference herein.

### BACKGROUND OF THE INVENTION

1. Field of the Invention

This invention relates to pharmaceutical vials. More particularly, this invention relates to vials that employ a needleless access to withdraw the contents thereof.

2. Description of the Background Art

Standard vials to contain and dispense parenteral pharmaceuticals have been in clinical use for many years. National & international standards for the configuration of these vial containers presently exist to standardize the shape of the vial at the seal and cap interface. The current practice is to pierce an elostomeric seal to gain access to and withdraw the vial contents in the clinical environment.

Many devices for the extraction of parenteral pharmaceuticals from traditional glass or plastic vials are presently in use worldwide. These devices range from a common needle on a medical syringe, to specialty vial caps that are attached to the vial to permit access with a syringe or other connection without using the obviously hazardous needle, to an array of specialized spiked devices with and without venting or other arrangements.

The present practice for the extraction of medicants from traditional vial containers requires the practitioner to remove either a frangible metal tab from the crimped on cap of the vial, (usually aluminum or plastic), or remove a full coverage cap to expose the elostomeric vial seal underneath. Access to the material within the vial is then accomplished by means of a metal needle on a syringe or other device, or by opening a pre-packaged vial access cap and attaching it to the vial. This separate vial access cap has an integral spike feature to pierce the elostomeric seal of the vial package in much the same manner as the metal needle.

Obviously the use of the needle on syringe presents the continual risk of injury and infectious contamination to the clinician, as well as requiring appropriate disposal of the needle after use. The integral spike on the “add-on” vial cap access device suffers from the distance it must protrude past the elostomeric vial seal, rendering it incapable of permitting extraction of the entire pharmaceutical contents of the vial. This results in waste from either the practice of over-filling the vials by pharmaceutical packagers, or by disposal of unused contents of the vial by the clinician. The only apparent alternative to extract the complete contents of the vial is to revert to use of the hazardous needle.

Use of the separate vial access cap attachment, results in waste from its single use on each vial, or the unacceptable risk of cross contamination from attempting use of the device on another vial. Attempting to re-sterilize single use vial access caps presents the risk of damage to the product, rendering it unsuitable or hazardous for use. The “add-on” cap can become structurally weakened from the sterilization process, resulting in failure of its integral spike feature when attempting to access the vial elostomeric seal, or worse, breaking off within the vial rendering the vial contents unusable without the risk of transmission of fractured polymer particulates to the patient. Additionally, the separate vial access product requires pre-packaging and sterilization, forcing the clinician to manipulate both opening the vial cap seal cover and opening the “add-on” cap package, and disposal, (waste) of the packaging.

One type of “add-on” cap package is taught by U.S. Pat. No. 6,695,829, the disclosure of which is incorporated by reference herein, that employs a piercing member internally of the cap that pierces the elostomeric seal of the vial once the syringe is fitted to the cap. However, like many of the prior art devices, the “add-on” cap package taught by U.S. Pat. No. 6,695,829 is principally limited to single dose vials.

Therefore, it is an object of this invention to provide an improvement which overcomes the aforementioned inadequacies of the prior art devices and provides an improvement which is a significant contribution to the advancement of the pharmaceutical vial art.

Another object of the invention is to provide a needleless access valve for vials that opens and closes upon the insertion and withdrawal of the neck of a standard syringe thereto.

Another object of the invention is to provide a needleless access valve for vials having a luer configuration that allows the fitting of a syringe having a standard luer connection thereto.

Another object of the invention is to provide a needleless vial that is configured to allow the syringe to withdraw of all of the contents of the vial.

Another object of the invention is to provide a needleless access vial that is economical to manufacture, assemble and sterilize.

Another object of the invention is to provide a needleless access vial that may be repeatably accessed and is therefore not limited to single use applications.

The foregoing has outlined some of the more pertinent objects of the invention. These objects should be construed to be merely illustrative of some of the more prominent features and applications of the intended invention. Many other beneficial results can be obtained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### SUMMARY OF THE INVENTION

For the purpose of summarizing this invention, this invention comprises a needleless access vial having a valve element with a compressible stem that seals into a valve body. The combination valve body and valve element is seated onto an annular ring integrally formed on the upper neck of the vial. During assembly, a retainer is positioned over the valve body and valve element and over the annular ring. The ring is then crimped under the annular ring of the vial to secure such components together.

The invention incorporates the needleless functionality directly into the pharmaceutical vial packaging, thereby eliminating the need for a separate “add-on” vial access cap as taught by the prior art. Yet, the invention also allows for the use of a needle if required.

The needleless access vial of the invention includes an internal luer configuration that allows the fitting of a syringe having a standard slip luer connection thereto. Moreover, the valve element of the invention is configured to allow repeated access. The needleless access vial is therefore not limited to single use vials. Finally, the needleless access vial is configured to allow the syringe to withdraw of all of the contents of the vial, thereby obviating the need to provide an extra volume of medicants in the vial to assure that the proper amount of medicants may be dispensed.

The design of the invention allows for ease in manufacturing, assembly and filling using established automated assembly, vial filling, packaging and sterilization equipment. Accordingly, the invention is characterized as incorporating needleless functionality directly into vial packaging that is simplistic in design and manufacture and results in easy adoption by pharmaceutical packagers into current automation equipment.

The foregoing has outlined rather broadly the more pertinent and important characteristics and features of the present invention in order that the detailed description of the invention that follows may be better understood so that the present contribution to the art can be more fully appreciated. Additional features of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a fuller understanding of the nature and objects of the invention, reference should be had to the following detailed description taken in connection with the accompanying drawings in which:

FIG. 1 is a side elevational view of the first embodiment of the needleless access vial of the invention including a needleless valve coupled with a pharmaceutical vial;

FIG. 2 is an exploded view of FIG. 1 showing the valve element positioned between the valve body and the upper annular ring of the vial;

FIG. 3 is a cross-sectional view of FIG. 2;

FIG. 4 is a enlarged cross-sectional view of the first embodiment of the needleless access vial of the invention;

FIG. 5 is an exploded isometric view of the second embodiment of the needleless access vial of the invention;

FIG. 6 is a side elevational view of FIG. 5;

FIG. 7 is a enlarged cross-sectional view of the second embodiment of the needleless access vial of the invention; and

FIGS. 8A, B & C are sequential views of the second embodiment of the needleless access vial of the invention showing the insertion of the neck a syringe therein for luer twist-locking therewith.

Similar reference characters refer to similar parts throughout the several views of the drawings.

### DETAILED DESCRIPTION OF THE PREFERRED EMBODIMENT

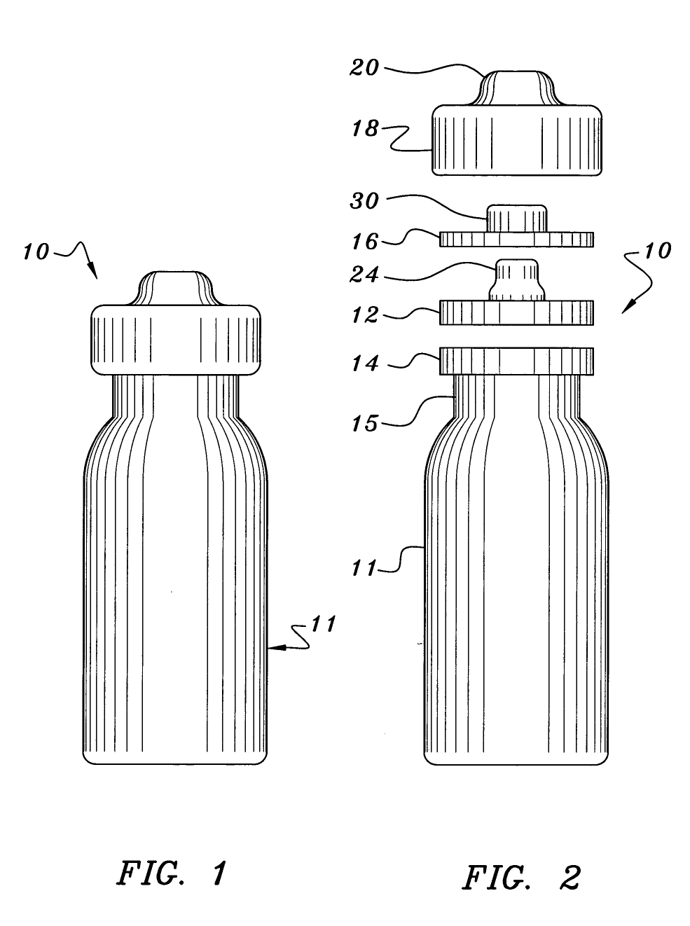

As shown in FIGS. 1-4, the first embodiment of the needleless access vial 10 of the invention comprises a pharmaceutical vial 11 sealed by a valve element 12.

As shown in FIG. 2, the needleless access via 10 comprises a valve element 12 positioned between an annular ring 14 integrally formed on the upper neck 15 of the vial 11. The vial 10 further comprises a valve body 16 positioned on the valve element 12. A retainer 18 is configured to be mounted over the sandwiched valve body 16, valve element 12 and annular ring 14 and then crimped under the annular ring 14 of the vial 11 to secure such components together. The upper dome 20 of the retainer 18 is perforated about its periphery allowing it to be easily removed to expose the valve body 16.

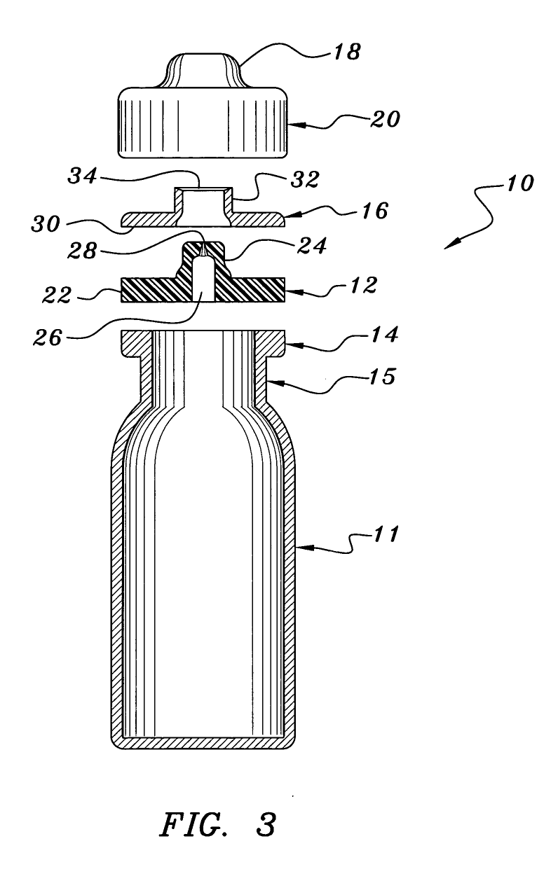

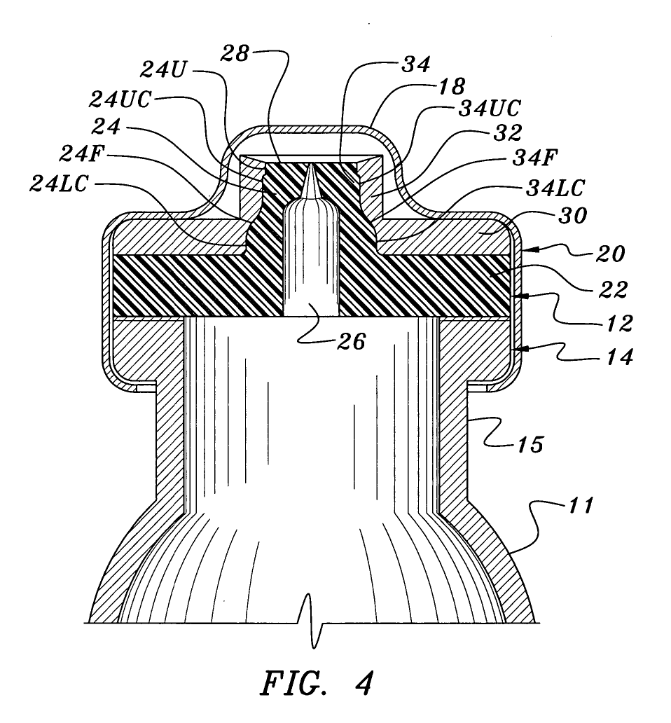

As shown in FIGS. 3 and 4, the valve element 12 comprises a disk portion 22 having an integral upstanding concentric center stem 24. The stem 24 includes a lower cylindrical portion 24LC, a frustro-conical portion 24F and an upper cylindrical portion 24UC. The upper rim of the stem 24 comprises a reduced-diameter notch or undercut 24U.

The valve element 12 further comprises a blind central passageway 26 with a slit 28 formed in the blind end. The uppermost blind end of the central passageway 26 is preferably dome-shaped. Slit 28 comprises a through-slit formed during manufacturing. Alternatively, slit 28 may comprise a blind slit formed during manufacture with a thin covering membrane serving as a mechanical microbial barrier to prevent any intrusion of microbes into the lumen of the slit 38 until such time as the vial 12 is first accessed as described hereinafter to burst the membrane.

The valve body 16 comprises a disk portion 30 having a concentric upstanding boss portion 32 having a central passageway 34 therethrough. The central passageway 34 is complementarily configured with a lower cylindrical portion 34LC, a frustro-conical portion 34F and an upper cylindrical portion 34UC to sealingly receive the lower cylindrical portion 24LC, frustro-conical portion 24F and upper cylindrical portion 24UC, respectively, of the center stem 24 of the valve element 12 when the stem 24 is inserted into the central passageway 34 during assembly.

Preferably, the inside diameters of the upper cylindrical portions 24UC and 34UC of the stem 24 and central passageway 34 are dimensioned relative to the slit 28 such that the slit 28 of the valve portion 24 is forced closed when the stem 24 is inserted into the central passageway 34 during assembly and during opening and closing of the valve as described hereinafter.

During use, the dome portion 20 of the retainer 18 is removed to allow access to the boss portion 32 of the valve body 16 and the valve portion 24 of the valve element 12 positioned in the central passageway 34 thereof. After swabbing to assure sterility, the neck of a syringe may then be inserted into the central passageway 34 to compress the stem 24 inwardly along the central passageway 34. As the stem 24 is forced inwardly along the central passageway 34 by the neck of the syringe, the neck of the syringe enters the slit 28 first causing the thin membrane if present to burst and then forcing the opposing edges of the slit 28 to spread apart to open allowing entry of the neck of the syringe.

Preferably, the upper cylindrical portion 34UC of the central passageway 34 of the valve body 16 comprises a conventional slip luer configuration to slip-receive conventional luer-configured necks of syringes. The luer tip of the syringe thus mates with the luer passageway 34 to form a fluid-tight connection between the syringe and the vial 12. The syringe is thus in fluid communication with the inside of the vial 12 and the medicants contained therein may be withdrawn into the syringe.

Preferably, the valve element 12 is composed of an elostomeric material. Upon removal of the syringe, the memory of the elostomeric material of the valve element 12 causes the stem 24 to return to its sealed position within the central passageway 34 of the valve body 16. The undercut 24U minimizes the tendency for the upper edge of the stem 24 to roll as it returns by virtue of its memory to its “at rest” position in the central passageway 34 of the valve body 16.

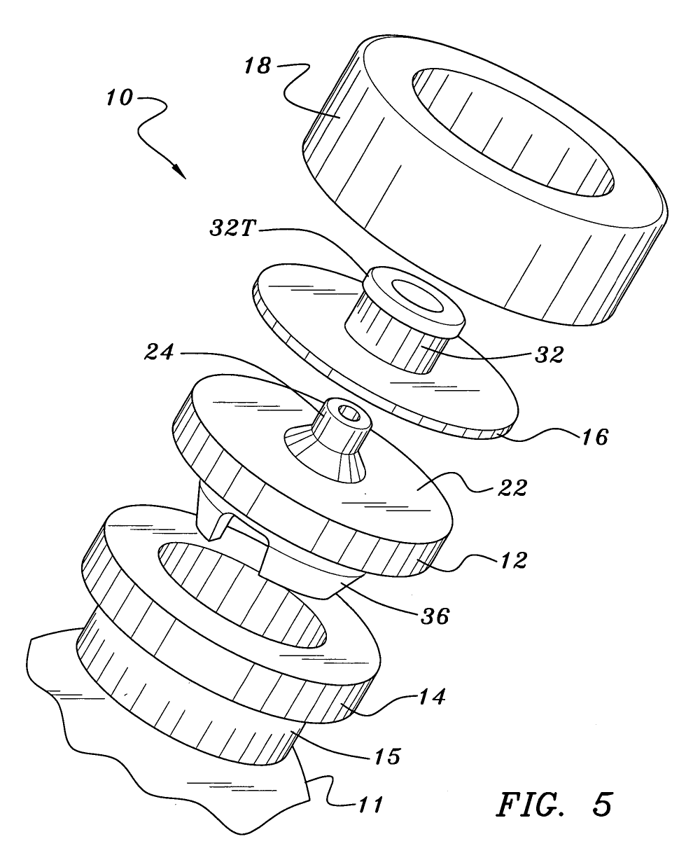

The second embodiment of the needleless access vial 10 of the invention is shown in FIGS. 5-8. This second embodiment is similar to the first embodiment. Therefore, for clarity, similar components are numbered with the same reference numerals.

More particularly, the second embodiment of the needleless access vial 10 comprises a valve element 12, valve body 16 and annular ring 14 sealingly sandwiched together by a retainer 18 with an optional removable dome 20. The valve element 12 comprises a disk portion 22 with a concentric center stem 24. However, the stem 24 of the second embodiment employs only the frustro-conical portion 24F and the upper cylindrical portion 24UC and not the lower cylindrical portion 24LC as employed in the first embodiment. Correspondingly, the central passageway 26 of the valve body 16 comprises only the frustro-conical portion 26F and the upper cylindrical portion 26UC and not the lower cylindrical portion 26LC as employed in the first embodiment. Further, the longitudinal length of the upper cylindrical portions 24UC & 26UC of the stem 24 and passageway 26 are of increased length. Finally, instead of or in addition to employing an undercut 24U as in the case of the first embodiment, the second embodiment of the stem 24 may protrude slightly beyond the central passageway 34.

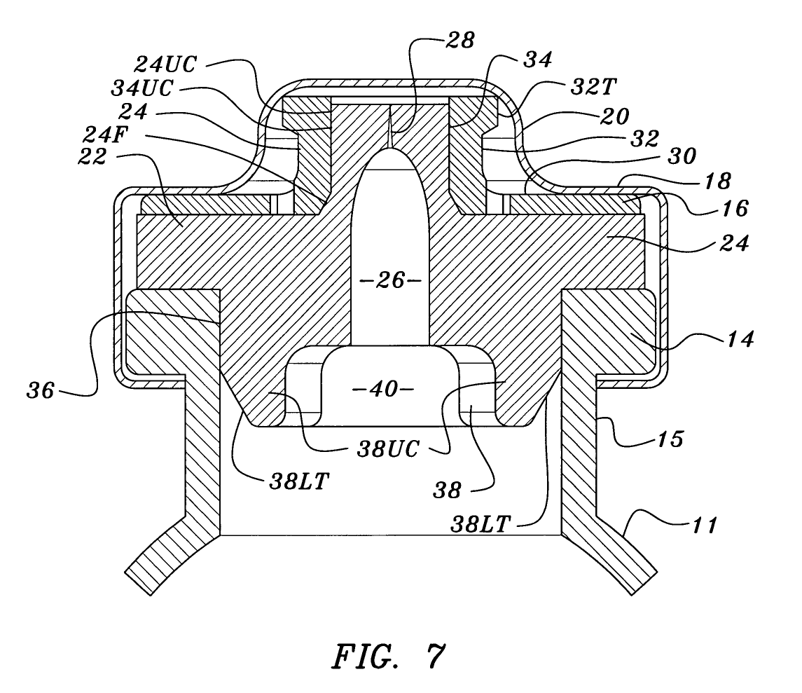

As best shown in FIG. 7, in addition to the upstanding boss portion 32 of the valve element 12, the second embodiment of the vial 10 comprises a downwardly-extending boss 36 that includes an outer diameter approximately equal to or slightly greater than the inner diameter of the neck of the vial 11. Further, the boss 36 comprises opposing arcuate legs 38 defined by transverse slot 40. Preferably, each leg 38 includes an upper cylindrical portion 38UC of the same diameter of the boss portion 36 and an lower inwardly-tapered portion 38T. Upon insertion of the neck of the syringe to force the stem 24 inwardly, the boss 36 provides increased sealing with the lumen of the neck of the vial 11 whereas, due to the taper 38T, the legs 38 are allowed to move radially outwardly toward the lumen.

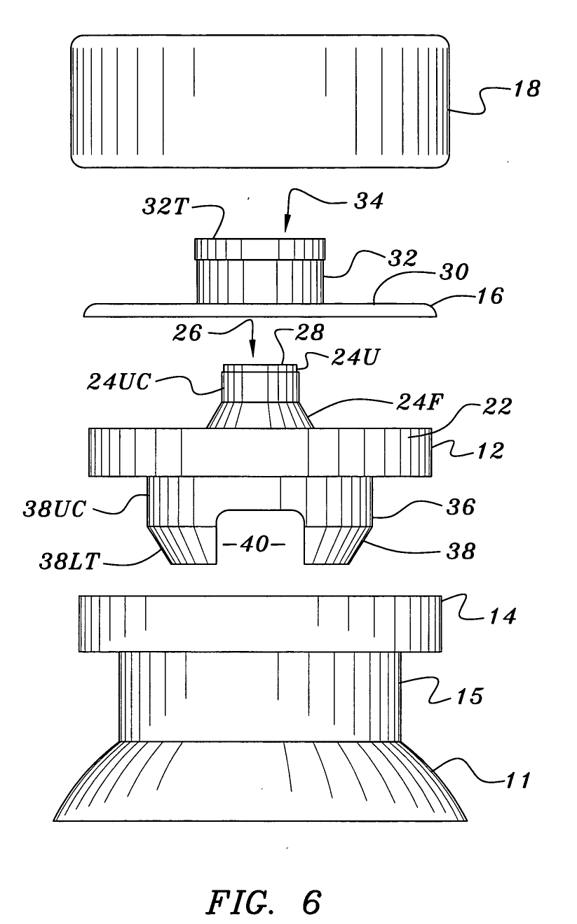

As best shown in FIGS. 6 & 7, the upper edge of the boss portion 32 of the valve body 16 may include a conventional luer thread 32T for connection to a corresponding conventional female luer on the syringe or other device to be connected therewith.

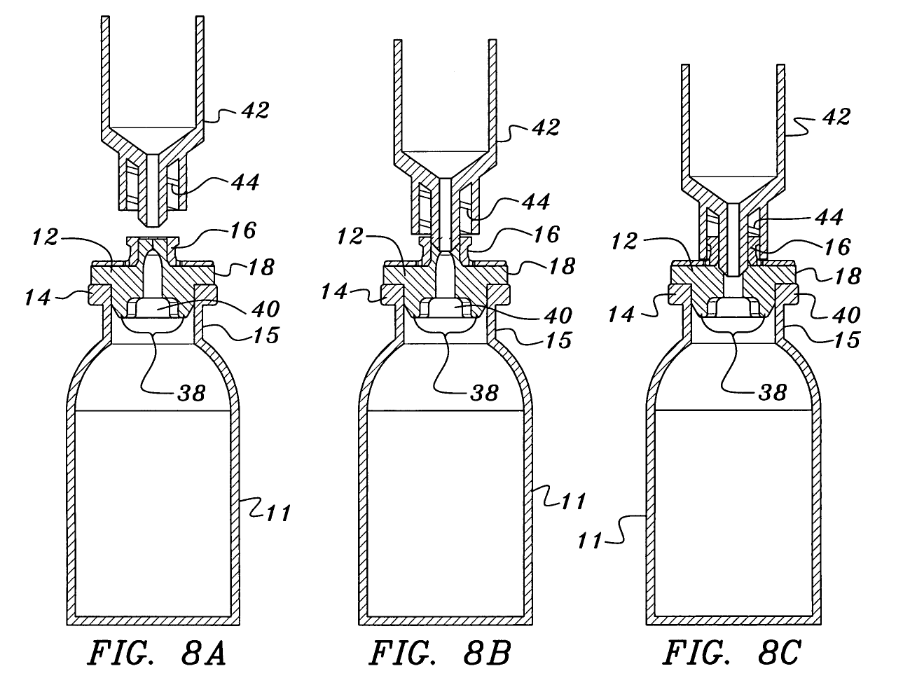

Referring now to FIGS. 8A, 8B & 8C, the vial 10 of the invention is particularly adapted to be accessed by a conventional syringe 42 having a female luer fitting 44 about its neck 46. As shown in FIG. 8A, the neck 46 of the syringe 42 is aligned with the stem 24 of the vial 10 of the invention. As shown in FIG. 8B, the neck 46 is inserted into the central passageway 34 to force the stem 24 inwardly. Concurrently, the luer thread 32T is engaged by the luer fitting 44. Upon twisting of the syringe 42 to engage the same, the stem 24 is forced fully inwardly as shown in FIG. 8C. In the full inward position, the slit 28 is forced open by the neck 46 so that the neck 46 is now in fluid communication with the contents of the vial 11.

An important feature of the invention is the inclusion of the slot 40 that allows the fluid contents of the vial 11, when inverted to withdraw the contents thereof, to flow around the lower tapered portion 38T of the arcuate legs 38 and into the central passageway 34 and then into the syringe 42. The entire contents of the vial 11 may be withdrawn since no pooling or trapping of the fluid contents occurs.

The present disclosure includes that contained in the appended claims, as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

Now that the invention has been described,

1. A needleless access vial, comprising in combination:
a vial;
a valve for sealing the vial, said valve comprising a valve element positioned in a valve body;
a retainer positioned about said valve body, valve element and a portion of said vial to sealingly sandwich said valve element between said portion of said vial and said valve body; and
said valve element comprising a central passageway including a slit formed therein for receiving a neck of an access device such as a syringe.

2. The needleless access valve as set forth in claim 1, wherein said portion of said vial comprises an annular ring of an upper neck portion of said vial.

3. The needleless access valve as set forth in claim 1, wherein said valve element comprises a disk portion having an upstanding concentric center stem that fits into a central passageway formed in said valve body.

4. The needleless access valve as set forth in claim 3, wherein said stem includes a frustro-conical portion and an upper cylindrical portion and wherein said central passageway correspondingly includes a frustro-conical portion and an upper cylindrical portion.

5. The needleless access valve as set forth in claim 4, wherein said stem further comprises a lower cylindrical portion and wherein said central passageway correspondingly includes a lower cylindrical portion.

6. The needleless access valve as set forth in claim 4, wherein a longitudinal length of said upper cylindrical portions of said stem and said passageway are of increased length.

7. The needleless access valve as set forth in claim 3, wherein an upper rim of said stem comprises a reduced-diameter undercut.

8. The needleless access valve as set forth in claim 1, wherein said central passageway comprises a blind central passageway and further including a slit formed in a blind end of said central passageway.

9. The needleless access valve as set forth in claim 1, wherein said slit comprises a through-slit.

10. The needleless access valve as set forth in claim 1, wherein said slit comprises a blind slit formed with a thin covering membrane that is burst when the valve element is first accessed.

11. The needleless access valve as set forth in claim 3, wherein said valve body comprises a disk portion having a concentric upstanding boss portion having a central passageway therethrough for receiving said stem.

12. The needleless access valve as set forth in claim 4, wherein said valve body comprises a disk portion having a concentric upstanding boss portion having a central passageway therethrough for receiving said stem, said central passageway of said boss having a frustro-conical portion and an upper cylindrical portion to sealingly receive said frustro-conical portion and said upper cylindrical portion, respectively, of said stem when said stem is inserted into said central passageway of said valve body.

13. The needleless access valve as set forth in claim 4, wherein said valve body comprises a disk portion having a concentric upstanding boss portion having a central passageway therethrough for receiving said stem, said central passageway of said boss having a lower cylindrical portion, a frustro-conical portion and an upper cylindrical portion to sealingly receive said lower cylindrical portion, said frustro-conical portion and said upper cylindrical portion, respectively, of said stem when said stem is inserted into said central passageway of said valve body.

14. The needleless access valve as set forth in claim 12, wherein an inside diameter of said upper cylindrical portions of said stem and said central passageway are dimensioned relative to said slit such that said slit is forced closed when said stem is inserted into said central passageway.

15. The needleless access valve as set forth in claim 1, wherein said valve body comprises a slip luer fitting.

16. The needleless access valve as set forth in claim 1, wherein said valve body comprises a twist luer fitting.

17. The needleless access valve as set forth in claim 3, wherein said valve element is composed of an elostomeric material having memory cause said stem to return to its sealed position within said valve body.

18. The needleless access valve as set forth in claim 1, wherein said retainer comprises a removable dome.

19. The needleless access valve as set forth in claim 1, further including a downwardly-extending boss with opposing legs defined by a transverse slot.

20. The needleless access valve as set forth in claim 19, wherein said legs comprise an inwardly tapered configuration.
